(19) United States
(12) Patent Application Publication
(10) Pub. No.: US 2006/0149389 A1
(43) Pub. Date: Jul. 6, 2006

(54) Corrective element for the articulation between the femur and the pelvis

(76) Inventor: Sergio Romagnoli, Vezzi Portio (IT)

Correspondence Address: Modiano & Associati;Via Meravigli 16, 20123 Milano, Europe, IT

(21) Appl. No.: 10/544,842

(22) PCT Filed: Feb. 12, 2004
(86) PCT No.: PCT/EP04/01306

§ 371 Date: Aug. 8, 2005

(30) Foreign Application Priority Data
Feb. 14, 2003 (IT) MI2003A000274

(51) Int. Cl. A61F 2/32; A61F 2/36; A61F 2/30

(52) U.S. Cl. 623/23.12; 623/22.11; 623/14.12

(57) ABSTRACT

A corrective element for the articulation between the femur and the pelvis, comprising a contoured body that can be inserted, without dislocation of the head of the femur, in the iliac acetabular region after incision of the articular capsule; the contoured body has a smooth outer surface that is substantially adapted to the iliac acetabular region and an inner surface that forms a seat for accommodating the femur head.

### TECHNICAL FIELD

The present invention relates to a corrective element or implant for the articulation between the femur and the pelvis.

### BACKGROUND ART

As is known, when problems in the articulation between the femur and the pelvis arise, currently the head of the femur is extracted from the acetabular seat and a prosthesis is inserted, by way of the most disparate technologies, recreating in practice the seat for accommodating the head of the femur.

This approach is particularly traumatic, since dislocation of the head of the femur causes considerable problems and further entails performing long and complex surgery.

### DISCLOSURE OF THE INVENTION

The aim of the invention is to eliminate the drawbacks noted above, by providing a corrective element for the articulation between the femur and the pelvis that allows to restore correct articulation without having to dislocate the head of the femur.

Within this aim, an object of the invention is to provide a corrective element that can be positioned in situ rapidly and easily, allowing to use a surgical method that is not invasive and reduces all the negative side effects linked to conventional surgery.

Another object of the present invention is to provide a corrective element for the articulation between the femur and the pelvis that thanks to its particular constructive characteristics is capable of giving the greatest assurances of reliability and safety in use.

Another object of the present invention is to provide a corrective element for the articulation between the femur and the pelvis that can be obtained easily starting from commonly commercially available elements and materials and is advantageously competitive from a merely economical standpoint.

This aim and these and other objects that will become better apparent hereinafter are achieved by a corrective element for the articulation between the femur and the pelvis, according to the invention, characterized in that it comprises a contoured body that can be inserted, without dislocation of the head of the femur, in the iliac acetabular region after incision of the articular capsule, said contoured body having a convex smooth outer surface that reproduces the acetabular shape and a concave smooth inner surface that forms the seat for accommodating said femur head.

This implant remains free and is not fixed by primary or secondary fixation. Its shape utilizes the load resultant and self-centers and is kept stable by the contact surfaces (head-femur-acetabulum) by muscle tension and by the articular capsule.

### BRIEF DESCRIPTION OF THE DRAWINGS

Further characteristics and advantages of the invention will become better apparent from the description of a preferred but not exclusive embodiment of a corrective element for the articulation between the femur and the pelvis, illustrated by way of nonlimiting example in the accompanying drawings, wherein:

FIG. 1 is a schematic perspective view of the corrective element according to the invention;

FIG. 2 is a sectional perspective view of the corrective element, taken from the other side;

FIG. 3 is a schematic side view of the step for the movement of the muscle bundles at the articulation between the femur and the pelvis;

FIG. 4 is a view of the initial step of insertion of the contoured body between the head of the femur and the acetabulum;

FIG. 5 is a schematic view of the contoured body after its insertion;

FIG. 6 is an enlarged-scale view of the contoured body arranged in the acetabulum and of the repositioned muscle bundles;

FIG. 7 is a plan view of the corrective element according to a further embodiment;

FIG. 8 is an elevation view of the corrective element of FIG. 7;

FIG. 9 is a sectional view of FIG. 7 taken along a median plane;

FIG. 10 is a plan view of the corrective element according to still another embodiment;

FIG. 11 is a sectional view of FIG. 10 taken along a median plane.

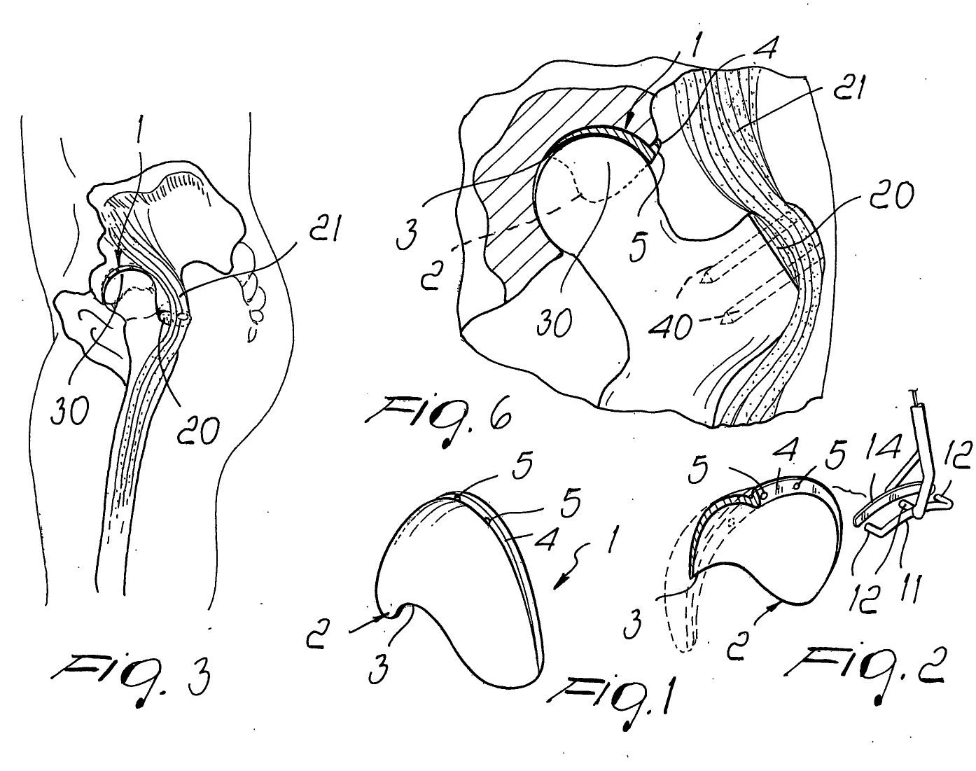

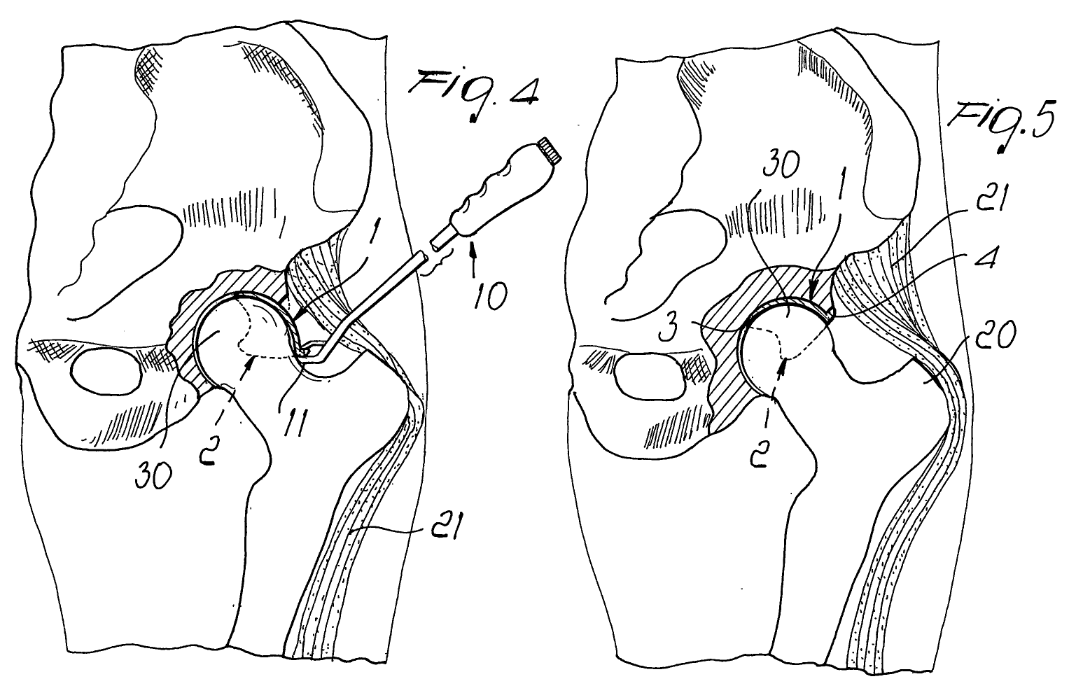

### WAYS OF CARRYING OUT THE INVENTION

With reference to the figures, the corrective element for the articulation between the femur and the pelvis, according to the invention, comprises a contoured body 1, which is preferably made of steel and has a shape that approximate a spherical portion.

The contoured body 1 has a beveled insertion edge 2, in which the thickness decreases to zero, and has, in a central portion, a central recess 3 for the reason explained hereinafter.

On the opposite side, the body 1 has a pushing and positioning edge 4, provided with multiple holes 5 for manipulation by means of a suitable instrument or lever 10 that facilitates insertion operations.

The instrument 10 has a push plate 11 that is provided with coupling pins 12 that enter the holes 5 located on the pushing edge 4, and there is also a complementary jaw 14, which is actuated by a suitable lever mechanism and engages the edge 4 on the opposite side, so as to practically clamp the edge 4 on the instrument 10 in order to allow to apply the force required for insertion. The edge 4 lies substantially at right angles to the surface of the body 1.

In practical use, in order to insert the corrective element, first the articular capsule is cut and optionally an osteotomy of the upper part 20 of the greater trochanter is performed, moving the muscle bundles, generally designated by the reference numeral 21.

This exposes the iliac acetabular region, and after cutting into the articular capsule, as shown schematically in FIG. 4, it is possible to begin the insertion of the contoured body, which in practice is fitted over the head of the femur, designated by the reference numeral 30, and is inserted in the acetabulum.

As insertion continues, the contoured body 1 is arranged so that it lies in close contact with the iliac acetabular region, since the contoured body has an outer surface that is shaped substantially complementarily to the iliac acetabular region and has an internal surface that forms a seat for accommodating the head of the femur 30.

This coupling in practice achieves self-positioning and self-centering of the corrective element in the acetabulum, accordingly achieving the possibility to restore the correct articulation without having to first dislocate the head of the femur.

Once positioning of the contoured body has been performed, the muscle bundles are repositioned and any portion of the greater trochanter that had been osteotomized is reconnected by using conventional surgical pins 40. Another surgical technique can also be carried out, i.e. without greater trochanter osteotomy and only with posterior or anterior capsulotomy.

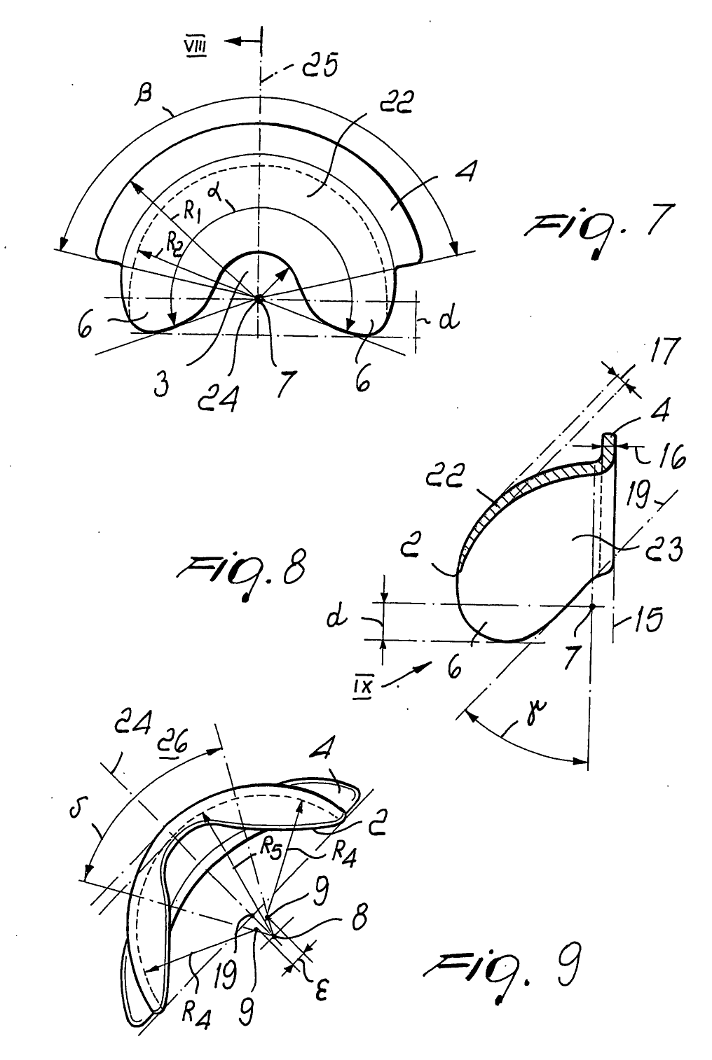

With reference to FIGS. 7, 8 and 9 an outer spherical surface 22 and an inner spherical surface 23 of the contoured (shell-like) spacer 1 terminate in an approximately equatorial plane 15 in correspondence with a pushing and positioning edge 4, which extends in the plane 15 only over a part of the periphery. If referred to a pole axis 24 of the pushing and positioning edge, the same extends through an angle β with a value from 130° to 180°. For the overall rigidity it is even more suitable for the value of β to be above 150°. Clear of the pushing and positioning edge 4 the outer and the inner spherical surfaces 23 and 22 are connected by an insertion edge 2, which in relation to the positioning edge 4 constitutes a central recess 3 around the pole region. In plan view (FIG. 7) it can be seen that the spherical surfaces 22 and 23, when related to the pole axis 24, describe an angle a larger than 180°. The angle a can exceed 210°. In the bisector of the angle β the contoured body or spacer 1 has a plane of symmetry 25, which divides the spacer into a left half and a right half. The pole axis 24 forms, as seen in the plan view, simultaneously a center 7 for the largest radius R₂ of the inner spherical face 23. With respect to the pole axis 24 the recess 3 possesses a minimum radius R₃, which, in comparison to the largest internal radius, corresponds to a percentage of 27% to 37%. In the left and right halves the insertion edge 2 extends a distance d past the pole axis 24 in the form of projecting ears 6, the distance d corresponding to 25% to 30% of the maximum inner radius R₂ of the inner spherical surface 23.

If referred to the pole axis the pushing and positioning edge 4 has an outer radius R₁ which corresponds to 120 to 140% of the radius R₂. The positioning edge 4 can have a thickness 16 from 1 to 5 mm. The two ears 6 are, as shown in FIG. 7, smoothly rounded. The actual edge between the inner and the outer spherical surfaces has a radius of at least 0.5 mm. The wall thickness 17 becomes smaller between the inner and outer spherical surfaces 23 and 22. In the remaining portion the wall thickness 17 may have values from 1 to 3.5 mm.

As shown in FIG. 8 the insertion edge 2, when considered in a projection perpendicular to the plane 25 of symmetry, extends away from the pushing and positioning edge 4 along a straight line 19, extending at an angle γ from the plane 15. The angle γ can be from 35 to 50°. If the angle γ is selected to be from 40° to 45°, insertion of the implant is still possible and furthermore a relatively large support surface is formed with the ears 6.

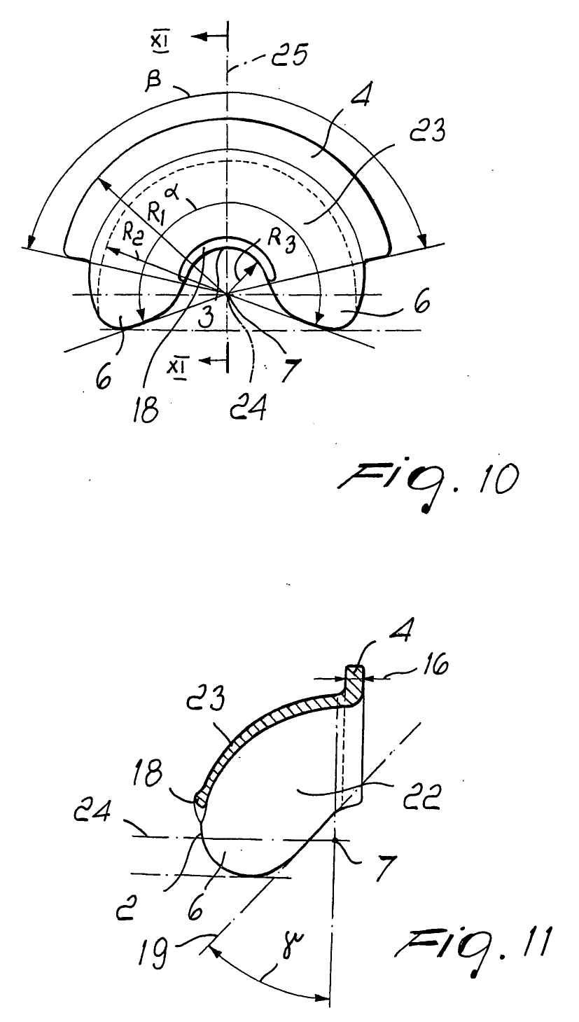

In accordance with the natural shape of a femur head it is advantageous for the inner spherical face 23 to have a flattened area in the eventual working direction. One form of such a flattened area is illustrated in FIG. 9. In the plane of the drawing two centers 9 are shown, which are arranged at a small distance ε apart and which respectively define (starting with the outer border), based on a radius R₄ the outline of the inner spherical face 23. In the median portion, which is characterized by an angle δ the outline is continued, on the basis of a larger radius R₅, to bridge over the distance ε, which may amount to 1 to 3 mm. The angle δ can lie from 40 to 70°. It is important that the transition from one curvature to the other curvature takes place continuously so that there is no irregularity in the curvature. Another possibility of providing a flattened area in the eventual working direction would be to provide smaller radiuses of curvature toward the pole. The spherical surface would then correspond to a section, cut in a very weak ellipsoid.

If it is assumed that the pushing and positioning edge 4 limits movements in relation to the acetabulum, then at least the inner spherical surface 23 should have a roughness of less than 0.1 μm.

The embodiment of FIGS. 10 and 11, which in its structure corresponds to the embodiment of FIGS. 7, 8 and 9, includes as a further feature a bead 18 projecting in the middle part of the central recess 3 toward the pole axis 24, and such bead may constitute a further security means to prevent accidental slipping out of place from the acetabulum. This could be an advantage in the case of a spacer 1 of a rubber-like material.

An application of the implant is in one case conceivable for elderly patients with local damage of the femur head cartilage or of the acetabulum cartilage. The implant would practically bridge over the defective area. A further application is merely as a placekeeper with the purpose of reducing pain. Elderly patients, who owing to the risk of a thrombosis, cannot be subjected to a major operation like the complete replacement of the hip joint could—more particularly if tied to a wheel chair—be freed of part of their pain, since the operation would rather be considered to be a minor one.

The selection of the material for the implant is therefore not limited at the outset. Rigid shells of a physiologically compatible metal alloy are conceivable, which have a low roughness Ra of less than 0.1 μm on their load bearing surfaces 23 and 22. The implant may also consist of a somewhat elastic physiologically compatible material. In the case of a merely placekeeper function with small movements without a load elastic, rubber-like but dimensionally stable plastics are conceivable.

Moreover plastics in the form of a hydrogel could be employed to provide inserts with a small wall thickness.

Coating of the load bearing surfaces 23 and 22 with a physiologically compatible anti-friction layer with the body is conceivable as well.

A further possibility is to endow the load bearing surfaces with a porosity like that of the natural meniscus in order to favor colonization with the own body cells.

From the above description it is therefore evident that the invention achieves the intended aim and objects, and in particular the fact is stressed that a contoured body is provided which has, in its front portion, a hollow that allows to preserve the round ligament and its vascularization, accordingly maintaining optimum conditions for its integration in the articulation without removing functional connections.

The contoured body can be manufactured in different sizes, depending on the anatomy of the patient, and can have various thickness, depending on the defect to be corrected; one should bear in mind that the inner and outer surfaces of the contoured body must be smooth, so as to allow its insertion without particular traumas, and that the beveled penetration edge must have a limited thickness both to facilitate its insertion and to avoid producing a dangerous discontinuity in the seat for accommodating the head of the femur.

The invention thus conceived is susceptible of numerous modifications and variations, all of which are within the scope of the appended claims.

All the details may further be replaced with other technically equivalent elements.

In practice, the materials used, so long as they are compatible with the specific use, as well as the contingent shapes and dimensions, may be any according to requirements.

The disclosures in Italian Patent Application No. MI2003A000274 from which this application claims priority are incorporated herein by reference.

What is claimed is:

1-23. (canceled)

24. A shell-like implant which is adapted to be inserted between a natural femur head and a natural acetabulum as a spacer, comprising an outer spherical surface, an inner spherical surface and a pushing and positioning edge lying in one plane and extending over a part of the periphery, and furthermore an insertion edge connecting the spherical surfaces, said edge, in relation to the positioning edge, constituting an open recess around the pole region.

25. The implant according to claim 24, wherein the pushing and positioning edge, as considered from above in the direction of a pole axis, describes an angle β from 130 to 180°.

26. The implant according to claim 24, wherein the pushing and positioning edge, as considered from above and in the direction of the pole axis, describes an angle β from 150 to 180°.

27. The implant according to claim 26, wherein the spherical surfaces, as considered from above in the direction of the pole axis, describe an angle α larger than 180°.

28. The implant according to claim 26, wherein the spherical surfaces, as considered from above in the direction of the pole axis, describe an angle α larger than 210°.

29. The implant according to claim 26, having, as considered from above in the direction of the pole axis, a plane of symmetry in a bisector of the angle β.

30. The implant according to claim 29, wherein the insertion edge, considered in its projection perpendicularly to the plane of symmetry, extends away from the pushing and positioning flange in a straight line with an angle γ from 35° to 50°.

31. The implant according to claim 29, wherein the insertion edge, considered in its projection perpendicularly to the plane of symmetry, extends away with an angle γ from 40° to 45°.

32. The implant according to claim 24, wherein the insertion edge has a minimum radius of 0.5 mm adjacent to a transition from the inner to the outer spherical surfaces.

33. The implant according to claim 25, wherein, as considered from above in the direction of the pole with the pole axis as the center, the recess has a minimum radius R3, which corresponds to 27 to 37% of the maximum inner radius R2 of the inner spherical surface.

34. The implant according to claim 33, wherein apart from the transition in the direction of the insertion edge a mean wall thickness between the inner and outer spherical surfaces in a range from 1 to 3.5 mm is kept.

35. The implant according to claim 33, wherein, as considered from above in the direction of the pole axis, an outer radius R1 of the pushing and positioning flange is corresponding to 120 to 140% of the radius R2 of the inner spherical surface.

36. The implant according to claim 33, wherein, as considered from above in the direction of the pole axis, the recess is enlarged by two ears projecting past the center, such ears projecting past the center by a distance d, the distance d having a size from 25 to 30% of the maximum inner radius R2 of the inner spherical surface.

37. The implant according to claim 24, wherein the pushing and positioning edge has a thickness from 1 to 5 mm.

38. The implant according to claim 33, wherein the inner spherical surface has a flattened area near the pole.

39. The implant according to claim 33, wherein in a plane perpendicular to the straight line the radius R5 of curvature of the inner spherical surface in the median region extending by an angle of δ is larger than the radiuses R4 of curvature in the two laterally adjacent regions, the centers of the two laterally adjacent regions differing by an amount of ε.

40. The implant according to claim 39, wherein the angle δ is from 40 to 70° and that the amount ε is from 1 to 3 mm.

41. The implant according to claim 33, wherein at least the inner spherical surface has a roughness Ra of less than 0.1 82 m.

42. The implant according to claim 24, made of a physiologically compatible metal alloy, or a physiologically compatible plastics, or of an elastic, rubber-like but dimensionally stable plastics.

43. The implant according to claim 42, consisting of a hydrogel.

44. The implant according to claim 43, wherein the bead consists of a hydrogel.

45. The implant according to claim 44, wherein at least the inner spherical surface is coated with an anti-friction layer.

46. The implant according to claim 24, wherein at least the inner spherical surface is porous and has pores which favor colonization with cartilage cells from the own body.
